(19) United States
(12) Patent Application Publication
(10) Pub. No.: US 2010/0217031 A1
(43) Pub. Date: Aug. 26, 2010

(54) PROCESS FOR THE PREPARATION OF ALKYL 3,3-DIALKOXYPROPIONATES

(76) Inventors: Wolfgang Wenger, Visp (CH); Cornelia Zur Täschler, Termen (CH); Daniel Zollinger, Sierre (CH)

Correspondence Address: HOFFMANN & BARON, LLP, 6900 JERICHO TURNPIKE, SYOSSET, NY, 11791, US

(21) Appl. No.: 12/679,045

(22) PCT Filed: Oct. 29, 2008
(86) PCT No.: PCT/EP2008/009128

§ 371 Date: Mar. 19, 2010

(30) Foreign Application Priority Data
Oct. 29, 2007 (EP) 07021098.4
Oct. 29, 2007 (EP) 07021104.0

(51) Int. Cl. C07C 67/46

(52) U.S. Cl. 560/183; 560/186

(57) ABSTRACT

The present invention relates to a continuous process for preparing alkyl 3,3-dialkoxy-propionates of the formula (RO)₂CHCH₂CO₂R, wherein R is C₁₋₆ alkyl, by reacting ketene with an ortho formate of formula (RO)₃CH in the presence of an acidic catalyst, characterized in that the reaction is carried out in a loop reactor.

The present invention relates to a continuous process for preparing alkyl 3,3-dialkoxypropionates of the formula (RO)₂CHCH₂CO₂R, wherein R is C₁₋₆ alkyl.

Alkyl 3,3-dialkoxypropionates are important C-3 building blocks, which themselves are intermediates for various products, such as pyrimidine, quinoline, uracil, fluvastatin, vitamin A and agrochemicals like the herbicide 1-methyl-5-hydroxypyrazole. One possible synthetic route for alkyl 3,3-dialkoxypropionates is the preparation from the corresponding orthoformate by reaction with ketene in the presence of an acidic catalyst. Thus, for example, G. Büchi prepares methyl 3,3-dimethoxypropionate in a yield of 19% at a reaction temperature of −70° C. (Büchi et al., J. Am. Chem. Soc. 1973, 95, 540-545). The preparation of ethyl 3,3-diethoxypropionate is described, for example, in U.S. Pat. No. 2,449,471 with a yield of 52% and in D. Crosby et al., J. Org. Chem. 1962, 27, 3083-3085 with a yield of 54%. In the references cited herein, the acidic catalyst used is the adduct of boron trifluoride and diethyl ether, and the reaction partners are reacted batch-wise. Since the reaction is highly exothermic and large amounts of ketene are difficult to handle, the batch-wise reaction is only possible for laboratory-scale batch sizes. Accordingly, it was an object of the present invention to provide an improved process suitable for preparing large amounts of alkyl 3,3-dialkoxypropionates in good yield and purity, which process is both without any risks and easy to carry out.

According to the invention, this object is achieved by the process as claimed in claim 1.

What is claimed is a continuous process for preparing alkyl 3,3-dialkoxypropionates of formula (RO)₂CHCH₂CO₂R, wherein R is C₁₋₆ alkyl, by reacting ketene (CH₂═C═O) with an ortho-formate of formula (RO)₃CH in the presence of an acidic catalyst, characterized in that the reaction is carried out in a loop reactor.

Here and as follows, the term “C₁₋₆ alkyl” is to be understood to mean any linear or branched alkyl group containing 1 to 6 carbon atoms. Examples of C₁₋₆ alkyl are methyl, ethyl, propyl, isopropyl, butyl, isobutyl, sec-butyl, tert-butyl, pentyl, isopentyl (3-methylbutyl), neopentyl (2,2-dimethylpropyl), hexyl, isohexyl (4-methylpentyl) and the like.

In a preferred embodiment, the orthoformate is selected from the group consisting of trimethyl orthoformate, triethyl orthoformate, tripropyl orthoformate and tributyl orthoformate. More preferably, the orthoformate is trimethyl orthoformate.

“Continuous operation” means that both the reaction partners and the reaction products are continuously added and removed, respectively. According to the invention, the ketene gas, the orthoformate and the acidic catalyst are continuously reacted with one another in a loop reactor.

Here, the term “loop reactor” does not denote a certain design, but only the principle of operation. In the most simple case, the loop reactor consists of a circularly closed tube (loop) equipped with a circulating pump. The loop has at least one connection for withdrawing a product stream and at least two connections for feeding the starting materials. The reaction can be carried out in a solvent or in the absence of a solvent. The acidic catalyst can be directly added into the reactor, or it can be mixed beforehand with the orthoformate and/or the solvent. The number and the positions of the feed connections have to be chosen accordingly. Preferably, the orthoformate is firstly mixed with the acidic catalyst and optionally with the solvent, whereupon the catalyst goes into solution or just forms a suspension. The resulting mixture is then fed into the loop reactor. In a particularly preferred process variant, the reaction is carried out in the absence of a solvent.

The ketene gas can be fed into the reaction mixture by any suitable gas distribution system, for example, by using a sparger, which is optionally provided with a frit or a nozzle. Preferably, the gaseous ketene and the liquid mixture consisting of orthoformate, catalyst and, optionally, solvent is introduced using a gas-liquid ejector. A gas-liquid ejector consists of different units described as follows. The liquid flow passes through a nozzle which generates a high velocity jet of fluid, thus creating suction of the ketene and entraining it into the ejector. Advantageously, the accelerated liquid-gaseous jet collides with the wall of an adjacent mixing tube, resulting in a rapid dissipation of kinetic energy. This forms an intensive mixing shock zone, in which the high turbulences produce a fine dispersion of bubbles. The ability to generate and finally disperse very small ketene bubbles into the liquid mixture leads to a favourable gas-liquid ratio of, for example, between 0.5 and 2.0, and to a much better dispersion of ketene in the liquid. The thus obtained two-phase mixture is finally injected into the fluid phase in the reactor, leading to an optimal efficacy in the subsequent chemical reaction. In addition, this way of gas distribution allows a consistent, pressure-free flowing of ketene into the gas-liquid ejector, which is particularly desired as ketene is prone to polymerization under pressure. Optionally, a swirl device directs, orientates and stabilizes the pumped liquid flow, before the liquid flow passes through the nozzle. A type of reactor as described above is also known as BUSS Loop® reactor.

Essentially, the reaction components are fed into the loop reactor in a simultaneous and continuous manner. This means that there are no major interruptions or strong variations in the molar ratio of the reactants within the reaction mixture. The circulation in the loop ensures good or even ideal mixing. However, it is not necessary to enforce an ideal mixing.

When simultaneously charging the reactants, a product stream is withdrawn from the loop reactor, in a volume which corresponds to the volume of the reactants charged, for subjecting to the following work-up procedure. This may take place, for example, via a simple overflow pipe or by pumping-off, while the pumping-off may be controlled using a level detector.

Depending on the feed rate, efficient cooling may be required due to the highly exothermic reaction. It can be achieved by known means, like by use of a cooling jacket covering a substantial part of the tube length or by a heat exchanger of conventional construction being incorporated into the loop.

The reaction is advantageously carried out at a temperature between −40° C. and 50° C. Optionally, the reactants may be pre-cooled before feeding into the loop reactor. Preferably, the reaction temperature is between −30° C. and 30° C., more preferably between −10° C. and 10° C.

The ketene used may be essentially pure or may contain inert gases, such as nitrogen, carbon monoxide and/or carbon dioxide, which advantageously are removed from the loop reactor by means of a suitable air-relief vent in order to prevent excessive pressure buildup.

The molar ratio of orthoformate to ketene is preferably between 0.9 and 1.2, more preferably between 1.0 and 1.1. These values refer to the amounts charged. The ratios actually present in the reaction mixture may differ more or less from these values.

In principle, each organic solvent in which the orthoformate is sufficiently soluble and which does not react with ketene or any other component can be used as solvent. Suitable solvents are, for example, aliphatic or aromatic hydrocarbons and ethers. However, it is also possible to dispense with a solvent, provided the used orthoformate is liquid. In a preferred embodiment, trimethyl orthoformate is directly reacted with ketene, i.e. without solvent, in the presence of an acidic catalyst.

The reaction can be catalyzed by all suitable acidic catalysts. Suitable acidic catalysts are both “classic” Lewis acids and “classic” Brönsted acids, and also acidic polysilicates.

Advantageously, the “classic” Lewis acids used are zinc(II) chloride, iron(III) chloride, aluminum chloride, boron trifluoride and its adducts with ethers, esters and similar compounds. A preferred adduct of boron trifluoride is the diethyl ether adduct. Preferred examples of “classic” Brönsted acids are sulfuric acid, phosphoric acid, methanesulfonic acid and benzenesulfonic acid.

Acidic polysilicates have Lewis and/or Brönsted acid properties and are therefore likewise suitable for the process according to the invention. The acidic polysilicates can also be employed in modified form or as mixtures. The formulae below are only given to illustrate the polysilicates but are not meant to be interpreted as a limitation. Suitable acidic polysilicates are, for example, amorphous polysilicates of the allophane type; chain polysilicates of the hormite type, such as “polygorskite”; two-layer polysilicates of the kaolin type, such as “kaolinite” Al₂(OH)₄[Si₂O₅] and “halloysite” Al₂(OH)₄[Si₂O₅]×2 H₂O; three-layer polysilicates of the smectite type, such as “sauconite” Na_{0.3}Zn₃(Si, Al)₄O₁₀(OH)₂×4 H₂O, “saponite” (Ca, Na)_{0.3}(Mg, Fe^{II})₃(Si, Al)₄O₁₀(OH)₂×4 H₂O, “montmorillonite” M_{0.3}(Al, Mg)₂Si₄O₁₀(OH)₂×n H₂O, wherein M in natural montmorillonite denotes one equivalent of one or more of the cations Na⁺, K⁺, Mg²⁺ and Ca²⁺, “vermiculite” (Mg, Fe^{II}, Al)₃(Al, SO₄O₁₀(OH)₂×4 H₂O, “nontronite” Na_{0.3}Fe₂^{III}(Si, Al)₄O₁₀(OH)₂×4 H₂O and “hectorite” Na_{0.3}(Mg, Li)₃Si₄O₁₀(F, OH)₂; three-layer polysilicates of the illite type; polysilicates having variable layers of the chlorite type and tectopolysilicates, such as zeolites, preferably of type Y in its H-form.

If required, the acidic polysilicates of the process according to the invention may be activated by treatment with acid and/or by treatment with a metal salt solution and/or by drying, and in the case of zeolites preferably by ion-exchange and/or by heating.

In a preferred embodiment, the catalysts used are acidic polysilicates of the smectite type and zeolites. A particularly preferred acidic polysilicate of the smectite type is montmorillonite, especially the types available under the names “montmorillonite K 10” and “montmorillonite KSF/0”, which are available, for example, from the company Süd-Chemie.

The acidic catalyst is advantageously employed in the process of the invention in an amount between 0.1% by weight and 20% by weight (based on orthoformate), preferably between 0.5 and 10% by weight. However, the amount depends on the activity of the catalyst and the reaction temperature.

When carrying out the reaction, it has to be ensured that the water content is as low as possible, since ketene and orthoformate may react with water in an unwanted manner.

Work-up is carried out by methods commonly known in the art and essentially depends on the physical properties of the formed alkyl 3,3-dialkoxypropionate and the other components of the reaction mixture. If a solid acidic catalyst is used, this is advantageously removed by filtration and the filtrate is worked up, whereas, if a liquid acid catalyst is used, this is first neutralized in the reaction mixture. The neutralization may be carried out, for example, by adding basic alkali metal salts, such as sodium hydroxide and potassium carbonate, or by adding alkali metal alkoxides, such as sodium methoxide and potassium ethoxide, or by adding similar basic reagents, such as anhydrous ammonia. Any precipitate may then be removed by filtration, and the filtrate can subsequently be purified, if required.

In a preferred embodiment, a solid acid catalyst is used which is filtered off in a first work-up step. The residue thus obtained is then either discarded or re-used in the reaction mixture as acidic catalyst, after its purification and optional re-activation if required.

After removal of the acidic catalyst, the filtrate is worked up in a known manner, preferably by distillation, to obtain the formed alkyl 3,3-dialkoxypropionate in neat form. In a particularly preferred embodiment, the unreacted orthoformate, which usually has a lower boiling point than the desired product, is distilled off after filtration and is then re-cycled into the reaction mixture, which significantly increases the total conversion of the reaction.

A further aspect of the present invention is the preparation of alkyl 3-alkoxyprop-2-enoates of formula ROCH═CHCO₂R from the alkyl 3,3-dialkoxypropionates of formula (RO)₂CHCH₂CO₂R which have been prepared according to the invention.

Alkyl 3-alkoxyprop-2-enoates are likewise important C-3 building blocks and are used, for example, for preparing alkyl 2,2,3-trichloro-3-alkoxypropionates, pyrazoles, furanones, thiophenes, aminothiazoles, isoxazole and vitamin A.

According to the invention, in a following step the alkyl 3,3-dialkoxypropionate, which has been formed as described above, is converted by means of heat supply and in the presence of an acid as catalyst into the corresponding alkyl 3-alkoxyprop-2-enoate of formula ROCH═CHCO₂R, wherein R is as defined above, by elimination of one molecule of the corresponding alcohol (ROH). Suitable acids are both liquid acids and solid acids, like acidic salts, acidically activated silica gel, acidic clay minerals, acidically activated carbon, acidic zeolites and cation exchange resins in their H-form. Optionally, the salts can be attached to carrier materials or can be modified.

Suitable acids are, for example, sulfuric acid, orthoboric acid, orthophosphoric acid, methanesulfonic acid, p-toluenesulfonic acid, sulfanilic acid, sodium bisulfate, phosphorus pentoxide, aluminum phosphate, zinc chloride, aluminum chloride and acidic zeolites. Particularly suitable are sulfuric acid, orthophosphoric acid, methanesulfonic acid, p-toluenesulfonic acid, sulfanilic acid, sodium bisulfate, phosphorus pentoxide, aluminum phosphate and acidic zeolites. Preferably, the amount of acid employed is between 0.05% by weight and 15% by weight (based on alkyl 3,3-dialkoxypropionate), particularly preferably between 0.1% by weight and 10% by weight.

The solvent used may be any solvent which does not react with the reaction components, such as, for example, ligroin. However, the elimination can also be carried out without solvent. Preferably, the reaction is carried out in the absence of a solvent.

Preferably, the elimination is carried out at a temperature between 50° C. and 250° C., more preferably between 80° C. and 200° C., and the reaction time is advantageously between 1 hour and 15 hours, preferably between 1 hour and 10 hours. Optionally, the reaction may also be carried out under reduced pressure. During elimination, the E-isomer of alkyl 3-alkoxyprop-2-enoate is formed with preference. Expediently, the formed alcohol (ROH) is directly distilled off during reaction.

After elimination, the alkyl 3-alkoxyprop-2-enoate obtained can be purified in a known manner, for example by rectification.

### EXPLANATION OF THE FIGURE

The appended schematic FIGURE as well as the examples serves only to illustrate the subject-matter of the invention without limiting it to these disclosures.

FIG. 1 shows, schematically, a device for the continuous preparation of alkyl 3,3-dialkoxypropionate. The specific meanings of the reference signs are as follows:

1. Feed of the mixture of orthoester and acidic catalyst

2. Ketene feed

3. Jet reactor

4. Circulating pump

5. Product removal

6. Heat exchanger

7. Nitrogen feed

8. Venting of inert gases

### EXAMPLES

The examples below illustrate embodiments of the invention. However, this is not meant to be construed as a limitation.

### Example 1

### Preparation of methyl 3,3-dimethoxypropionate

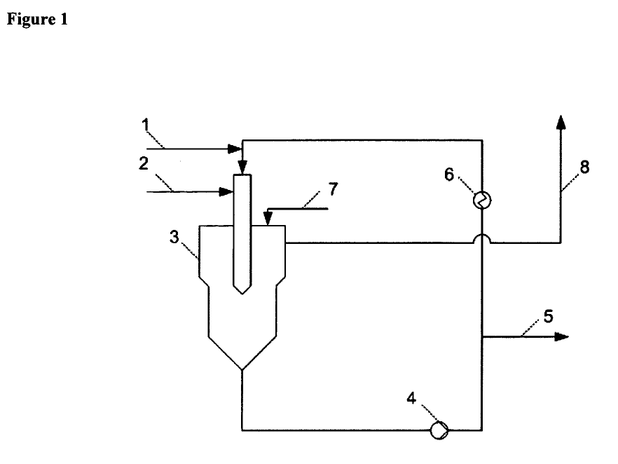

Simultaneously, but separately, 150 kg/h (1.413 kmol/h) of trimethyl orthoformate (Fluka), comprising 1.5% by weight of montmorillonite K10 (Süd-Chemie), and 84 kg/h of ketene (ketene content about 70%, remainder inert gases, such as N₂, CO and CO₂, i.e. neat ketene about 59 kg/h, that is about 1.4 kmol/h) were fed into a 620 L jet reactor (see FIG. 1), which had been inertized and cooled to an internal temperature of 0° C. Under an atmosphere of nitrogen, the reaction mixture was kept at a temperature of about 0° C. and circulated in the loop via a circulating pump. Corresponding to the amount of starting materials added, and also continuously, a corresponding part of the reaction mixture flowed over into a collecting tank. After filtration, the purity of the filtrate was determined by GC as 80% methyl 3,3-dimethoxypropionate, 8% unreacted trimethyl orthoformate, 4% methyl 3-methoxyprop-2-enoate and 4% methyl acetate.

By virtue of its low boiling point, it was easy to remove trimethyl orthoformate by distillation. The recovered starting material was subsequently re-cycled into the reaction mixture. The yield of methyl 3,3-dimethoxypropionate was 82% (based on conversion).

### Example 2

### Preparation of methyl 3-methoxyprop-2-enoate

Under an atmosphere of nitrogen, 0.2 g (2 mmol) of methanesulfonic acid (Fluka) was added to 150 g of a filtrate, analogously obtained as described in example 1 (about 85%, 0.86 mol of methyl 3,3-dimethoxypropionate), in a distillation apparatus with round-bottomed flask. Under constant flow of nitrogen, the mixture was slowly heated to 160° C., and the methanol formed was directly distilled off. After 6 hours, the heat supply was stopped. The methyl 3-methoxyprop-2-enoate obtained in this manner was 88% pure (GC) and was purified by rectification at 10 kPa. The yield was 85 g (85%) of methyl 3-methoxyprop-2-enoate (K_{10 kPa}=95° C.) with a purity of 99% (GC).

### Example 3

### Preparation of methyl 3-methoxyprop-2-enoate

The reaction was carried out analogously to example 2 using 5 g (content 99%, 34 mmol) of pure-distilled methyl 3,3-dimethoxypropionate and 25 mg (0.13 mmol) of p-toluenesulfonic acid monohydrate (Fluka). The resulting crude product had a content of 91% (GC) of methyl 3-methoxyprop-2-enoate.

### Example 4

### Preparation of methyl 3-methoxyprop-2-enoate

The reaction was carried out analogously to example 2 using 5 g (content 99%, 34 mmol) of pure-distilled methyl 3,3-dimethoxypropionate and 47 mg (0.27 mmol) of sulfanilic acid (Fluka). The resulting crude product had a content of 92% (GC) of methyl 3-methoxyprop-2-enoate.

### Example 5

### Preparation of methyl 3-methoxyprop-2-enoate

The reaction was carried out analogously to example 2 using 5 g (content 99%, 34 mmol) of pure distilled methyl 3,3-dimethoxypropionate and 31 mg (0.31 mmol) of orthophosphoric acid (Fluka). The resulting crude product had a content of 88% (GC) of methyl 3-methoxyprop-2-enoate.

### Example 6

### Preparation of methyl 3-methoxyprop-2-enoate

After distillative removal of the unreacted trimethyl orthoformate from example 1, 4.4 t (30 kmol) of the methyl 3,3-dimethoxypropionate thus obtained were reacted under an atmosphere of nitrogen with 6 kg (62 mol) of methanesulfonic acid analogously to example 2. Rectification at 10 kPa gave 2.4 t (21 kmol, 69% based on trimethyl orthoformate employed) of methyl 3-methoxyprop-2-enoate (K_{10 kPa}=95° C.) in a purity of 93% (GC).

1. A continuous process for preparing alkyl 3,3-dialkoxypropionates of formula (RO)₂CHCH₂CO₂R, wherein R is C₁₋₆ alkyl, by reacting ketene with an orthoformate of formula (RO)₃CH in the presence of an acidic catalyst, characterized in that the reaction is carried out in a loop reactor.

2. The process of claim 1, wherein the orthoformate is selected from the group consisting of trimethyl orthoformate, triethyl orthoformate, tripropyl orthoformate and tributyl orthoformate.

3. The process of claim 1, wherein the orthoformate is firstly mixed with the acidic catalyst and only then fed into the loop reactor.

4. The process of claim 1, wherein the loop reactor comprises a gas-liquid ejector (jet reactor).

5. The process of claim 1, wherein the reaction is carried out at a temperature between −40° C. and 50° C.

6. The process of claim 1, wherein the molar ratio of orthoformate to ketene is between 0.9 and 1.2.

7. The process of claim 1, wherein the process is carried out in the absence of a solvent.

8. The process of claim 1, wherein the acidic catalyst is a Lewis acid, a Brönsted acid or an acidic polysilicate.

9. The process of claim 8, wherein the Lewis acid is selected from the group consisting of zinc(II) chloride, iron(III) chloride, aluminum chloride, boron trifluoride and its adducts with ethers and esters.

10. The process of claim 8, wherein the Brönsted acid is selected from the group consisting of sulfuric acid, phosphoric acid, methanesulfonic acid and benzenesulfonic acid.

11. The process of claim 8, wherein the acidic polysilicate is selected from the group consisting of acidic, amorphous polysilicates of the allophane type; acidic, chain polysilicates of the hormite type; acidic, two-layer polysilicates of the kaolin type; acidic, three-layer polysilicates of the smectite type; acidic, three-layer polysilicates of the illite type; acidic, variable-layer polysilicates of the chlorite type; and acidic tectopolysilicates.

12. The process of claim 11, wherein the acidic, three-layer polysilicate of the smectite type is selected from the group consisting of sauconite, saponite, montmorillonite, vermiculite, nontronite and hectorite.

13. The process of claim 1, wherein the orthoformate is trimethyl orthoformate and the acidic catalyst is montmorillonite.

14. The process of claim 1, wherein the acidic catalyst is employed in an amount between 0.1% by weight and 20% by weight (based on orthoformate).

15. The process of claim 1, wherein in a following step the formed alkyl 3,3-dialkoxypropionate is converted by means of heat supply and in the presence of an acid into the corresponding alkyl 3-alkoxyprop-2-enoate of formula ROCH═CHCO₂R, wherein R is as defined above, by elimination of the corresponding alcohol (ROH).

16. The process of claim 15, wherein the acid is selected from the group consisting of sulfuric acid, orthophosphoric acid, methanesulfonic acid, p-toluenesulfonic acid, sulfanilic acid, sodium bisulfate, phosphorus pentoxide, aluminum phosphate and acidic zeolites.

17. The process of claim 16, wherein the acid is methanesulfonic acid.

18. The process of claim 15, wherein the acid is employed in an amount between 0.05% by weight and 15% by weight (based on alkyl 3,3-dialkoxypropionate).

19. The process of claim 15, wherein the process is carried out in the absence of a solvent.

20. The process of claim 15, wherein the reaction is carried out at a temperature between 50° C. and 250° C.

21. The process of claim 15, wherein the reaction time is between 1 hour and 15 hours.

22. Use of an alkyl 3,3-dialkoxypropionate as obtained according to claim 1 for preparing the corresponding alkyl 3-alkoxyprop-2-enoate.
